(19) United States
(12) Patent Application Publication
(10) Pub. No.: US 2024/0407796 A1
(43) Pub. Date: Dec. 12, 2024

(54) SHOCKWAVE VALVULOPLASTY CATHETER SYSTEM

(71) Applicant: Shockwave Medical, Inc., Santa Clara, CA (US)

(72) Inventors: Daniel HAWKINS, Fremont, CA (US); John M. ADAMS, Snohomish, WA (US)

(73) Assignee: Shockwave Medical, Inc., Santa Clara, CA (US)

(21) Appl. No.: 18/810,896

(22) Filed: Aug. 21, 2024

Related U.S. Application Data
(63) Continuation of application No. 17/231,936, filed on Apr. 15, 2021, now Pat. No. 12,102,342.
Application No. 17/231,936 is a continuation of application No. 16/183,438, filed on Nov. 7, 2018, now Pat. No. 11,000,299.
Application No. 16/183,438 is a continuation of application No. 15/213,105, filed on Jul. 18, 2016, now Pat. No. 10,149,690.
Application No. 15/213,105 is a continuation of application No. 14/693,155, filed on Apr. 22, 2015, now Pat. No. 9,421,025.
Application No. 14/693,155 is a continuation of application No. 12/611,997, filed on Nov. 4, 2009, now Pat. No. 9,044,618.
(60) Provisional application No. 61/111,600, filed on Nov. 5, 2008.

(51) Int. Cl. A61B 17/22; A61M 25/10; A61N 1/05; A61N 1/38

(52) U.S. Cl. CPC A61B 17/22022; A61B 17/22004; A61B 17/2202; A61B 2017/22025; A61B 2017/22051; A61B 2017/22054; A61B 2017/22061; A61B 2017/22062; A61B 2017/22068; A61B 2017/22098; A61M 25/1002; A61M 2025/1072; A61N 1/056; A61N 1/38

(57) ABSTRACT

A valvuloplasty system comprises a balloon adapted to be placed adjacent leaflets of a valve. The balloon is inflatable with a liquid. The system further includes a shock wave generator within the balloon that produces shock waves. The shock waves propagate through the liquid and impinge upon the valve to decalcify and open the valve.

### CLAIM OF PRIORITY

The present application is a continuation of U.S. patent application Ser. No. 17/231,936, filed Apr. 15, 2021, which is a continuation of U.S. patent application Ser. No. 16/183,438, filed Nov. 7, 2018, now issued as U.S. Pat. No. 11,000,299, which is a continuation of U.S. patent application Ser. No. 15/213,105, filed Jul. 18, 2016, now issued as U.S. Pat. No. 10,149,690, which is a continuation of U.S. patent application Ser. No. 14/693,155, filed Apr. 22, 2015, now issued as U.S. Pat. No. 9,421,025, which is a continuation of U.S. patent application Ser. No. 12/611,997, filed Nov. 4, 2009, now issued as U.S. Pat. No. 9,044,618, which claims the benefit of copending U.S. Provisional Patent Application Ser. No. 61/111,600, filed Nov. 5, 2008, each of which is incorporated herein by reference in its entirety.

### BACKGROUND OF THE INVENTION

Aortic calcification, also called aortic sclerosis, is a buildup of calcium deposits on the aortic valve in the heart. This often results in a heart murmur, which can easily be heard with a stethoscope over the heart. However, aortic calcification usually doesn't significantly affect the function of the aortic valve.

In some cases, though, the calcium deposits thicken and cause narrowing at the opening of the aortic valve. This impairs blood flow through the valve, causing chest pain or a heart attack. Doctors refer to such narrowing as aortic stenosis.

Aortic calcification typically affects older adults. But when it occurs in younger adults, it's often associated with an aortic valve defect that is present at birth (congenital) or with other illnesses such as kidney failure. An ultrasound of the heart (echocardiogram) can determine the severity of aortic calcification and also check for other possible causes of a heart murmur.

At present there is no specific treatment for aortic calcification. General treatment includes the monitoring for further developments of heart disease. Cholesterol levels are also checked to determine the need for medications to lower cholesterol in the hope to prevent progression of aortic calcification. If the valve becomes severely narrowed, aortic valve replacement surgery may be necessary.

The aortic valve area can be opened or enlarged with a balloon catheter (balloon valvuloplasty) which is introduced in much the same way as in cardiac catheterization. With balloon valvuloplasty, the aortic valve area typically increases slightly. Patients with critical aortic stenosis can therefore experience temporary improvement with this procedure. Unfortunately, most of these valves narrow over a six to 18 month period. Therefore, balloon valvuloplasty is useful as a short-term measure to temporarily relieve symptoms in patients who are not candidates for aortic valve replacement. Patients who require urgent noncardiac surgery, such as a hip replacement, may benefit from aortic valvuloplasty prior to surgery. Valvuloplasty improves heart function and the chances of surviving non-cardiac surgery. Aortic valvuloplasty can also be useful as a bridge to aortic valve replacement in the elderly patient with poorly functioning ventricular muscle. Balloon valvuloplasty may temporarily improve ventricular muscle function, and thus improve surgical survival. Those who respond to valvuloplasty with improvement in ventricular function can be expected to benefit even more from aortic valve replacement. Aortic valvuloplasty in these high risk elderly patients has a similar mortality (5%) and serious complication rate (5%) as aortic valve replacement in surgical candidates.

The present invention provides an alternative treatment system for stenotic or calcified aortic valves. As will be seen subsequently, the embodiments described herein provide a more tolerable treatment for aortic stenosis and calcified aortic valves than the currently performed aortic valve replacement. The invention also provides a more effective treatment than current valvuloplasty therapy.

### SUMMARY OF THE INVENTION

In one embodiment, a valvuloplasty system comprises a balloon adapted to be placed adjacent leaflets of a valve, the balloon being inflatable with a liquid, and a shock wave generator within the balloon that produces shock waves that propagate through the liquid for impinging upon the valve. The balloon may be adapted to be placed on opposite sides of the valve leaflets or within the valve annulus.

The system may further comprise an elongated tube. The balloon may be at the distal end of the elongated tube.

The balloon may include a first balloon chamber and a second balloon chamber. The first and second balloon chambers may be longitudinally spaced from each other.

The elongated tube may include a lumen. The first and second balloon chambers are in fluid communication with the elongated tube lumen.

The shock wave generator may comprise a first shock wave source within the first balloon chamber and a second shock wave source within the second balloon chamber. The first and second shock wave sources may comprise a first electrical arc generator and a second electrical arc generator. The electrical arc generators may comprise at least one electrode adapted for connection to a voltage pulse generator. Each of the electrical arc generators may comprise an electrode pair adapted for connection to a voltage pulse generator. Each of the electrode pairs may comprise a pair of coaxially arranged electrodes.

They may further comprise a high voltage catheter including the first and second electrical arc generators. The first and second electrical arc generators may be longitudinally spaced from each other for being received within the first and second balloon chambers, respectively.

As mentioned above, the balloon may be adapted to be placed within the valve annulus. To that end, the balloon may have a reduced diameter portion adapted to be received within the valve annulus.

The balloon may be formed of a compliant material.

Alternatively, the balloon may be formed of a non-compliant material.

According to another embodiment, a catheter system comprises an elongated carrier and a balloon carried by the elongated carrier. The balloon is arranged to receive a fluid therein that inflates the balloon. The system further includes at least one arc generator including at least one pair of coaxially arranged electrodes within the balloon that forms a mechanical shock wave within the balloon.

The system may further include a cable comprising a center conductor and an outer conductive shield insulated from the inner conductor. A first one of the coaxially arranged electrodes may be at least in part formed by the center conductor of the cable, and a second one of the coaxially arranged electrodes may be at least in part formed by the outer conductive shield of the cable.

According to a further embodiment, a valvuloplasty method for treating a valve having leaflets and an annulus comprises placing a balloon adjacent to the leaflets of the valve, inflating the balloon with a liquid, and producing shock waves within the balloon that propagate through the liquid for impinging upon the valve leaflets and the valve annulus.

The placing steps may be performed by placing the balloon on opposite sides of the valve leaflets. Alternatively the placing step may be performed by placing the balloon within the valve annulus.

### BRIEF DESCRIPTION OF THE DRAWINGS

The features of the present invention which are believed to be novel are set forth with particularity in the appended claims. The various described embodiments of the invention, together with representative features and advantages thereof, may best be understood by making reference to the following description taken in conjunction with the accompanying drawings, in the several figures of which like reference numerals identify identical elements, and wherein:

FIG. 1 is a cut away view of the left ventricle, the aorta, and the aortic valve of a heart showing a reduced aortic valve open area and thickened valve leaflets due to calcium and fibrotic tissue;

FIG. 2 is a cut away view of the aortic valve of a heart with a treatment balloon placed on both sides of the aortic valve leaflets, according to an embodiment of the present invention;

FIG. 3 is a schematic view of a dual shockwave balloon embodying the invention attached to a high voltage power supply; and

FIG. 4 is a cut away view of a heart showing an alternate valvuloplasty shock wave balloon according to a further embodiment and aspects of the present invention.

### DETAILED DESCRIPTION OF THE INVENTION

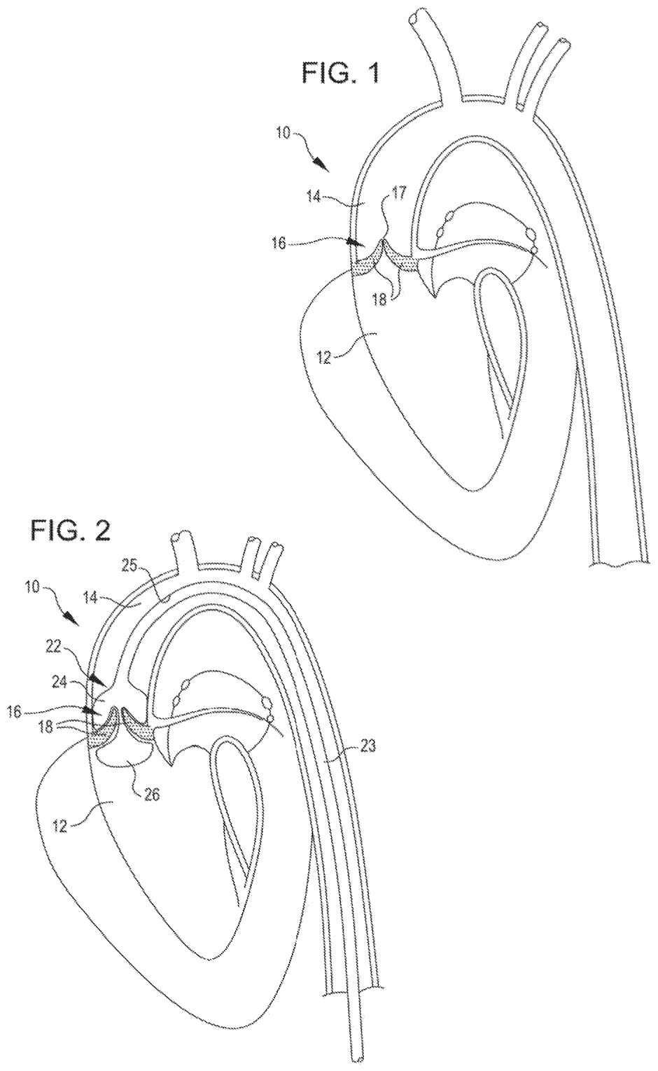

Referring now to FIG. 1, it is a cut away view of the left ventricle 12, the aorta 14, and the aortic valve 16 of a heart 10 with a stenotic and calcified aortic valve 16. Here more particularly, it may be seen that the opening 17 of the stenotic and calcified aortic valve 16 is restricted in size and that the valve leaflets 18 are thickened with calcium deposits and fibrotic tissue. The thickened leaflets 18 and smaller valve opening 17 restrict blood flow from the heart creating excess work for the heart 10 and poor cardiac output. As previously mentioned, current treatment includes replacement of the valve or attempts too stretch the valve annulus with a balloon.

FIG. 2 is a cut away view of the aortic valve 16 with a treatment balloon 22 placed on both sides of the aortic valve leaflets 18. The balloon 22 may be formed from a compliant or a non-compliant material. The balloon, as seen in FIG. 2, is at the distal end of an elongated tube 23. The treatment balloon 22 has two longitudinally spaced chambers 24 and 26 that share a common inflation lumen 25 of the tube 23. Alternatively the balloon chambers 24 and 26 may not share the same inflation fluid path. The chambers 24 and 26 are longitudinally spaced such that chamber 24 is positioned on one side of the aortic valve leaflets 18 and chamber 26 is positioned on the other side of the aortic valve leaflets 18. The chambers 24 and 26 are inflated with saline/contrast mixture, for example. Each chamber 24 and 26 may contain an electrode (as shall be seen subsequently) that can produce electrical arcs to deliver timed shock waves. The shock waves can be synchronized to concurrently impinge upon both sides of the leaflets 18 to maximize the effectiveness of breaking calcium deposits. Such shock waves may be generated and also synchronized to the R wave of the heart 10 in a manner as described for example in co-pending application No. 61/061,170 filed on Jun. 13, 2008, which application is incorporated herein in its entirety.

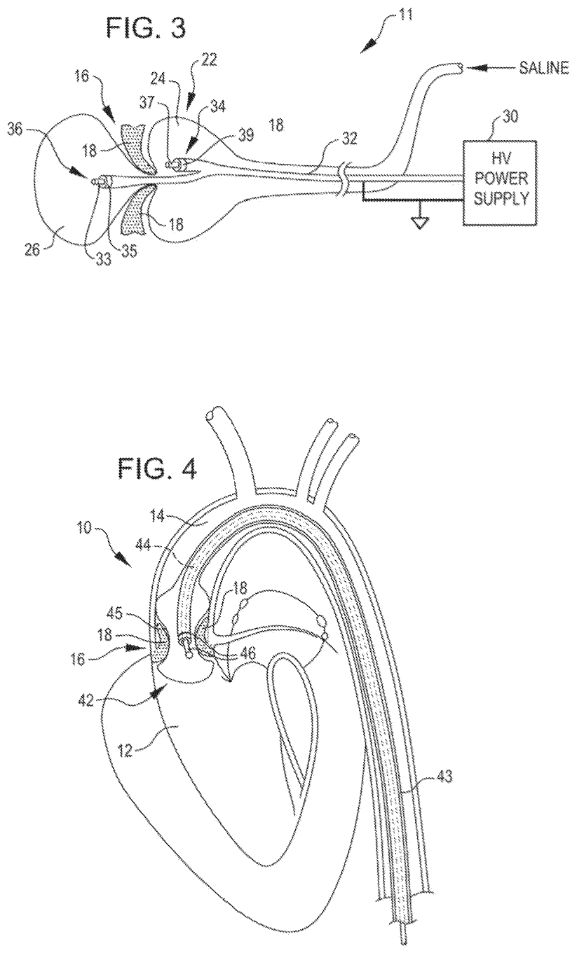

FIG. 3 is a schematic view of a valvuloplasty system 11 embodying the present invention. The system 11 includes the dual shockwave balloon 22. The balloon 22 has received a high voltage catheter 32 that is connected to a high voltage power supply 30. The schematic representation shows the positioning of the balloon chambers 24 and 26 above and below the leaflets 18 of the aortic valve 16. As previously described, shock waves will impinge upon opposite sides of the leaflets 18 to more effectively break calcium deposits in the valve leaflets 18. The annulus will also be treated in this arrangement. To that end, the high voltage catheter 32 includes electrode pairs 34 and 36 that are coaxially arranged electrodes placed in chambers 24 and 26 respectively of the balloon 22. More specifically, electrode pair 34 is at the distal end of a first cable and comprises a center conductor 33 and an outer conductive shield 35. Similarly, electrode pair 34 is at the distal end of a second cable and comprises a center conductor 37 and an outer conductive shield 39. High voltage pulses from power supply 30 are applied to the electrode pairs 34 and 36 in a manner as described in the aforementioned application Ser. No. 61/061,170 to create shockwaves within the fluid within the chambers 24 and 26 of the balloon 22. The shock waves impinge upon the valve leaflets 18 and the valve annulus to cause the break up of calcium deposits and fibrotic tissue on the valve leaflets 18 and annulus to open the aortic valve 16.

FIG. 4 shows an alternate valvuloplasty shock wave balloon 42 at the distal end of an elongated tube 43. The balloon 42 is placed in the annulus of the aortic valve 16. To that end, the balloon 42 has a reduced diameter portion 45 for being received within the valve annulus. The balloon 42 has a high voltage catheter 44 therein that terminates in an electrode pair 46. As in the previous embodiment, the electrode pair 46 may comprise a pair of coaxially arranged electrodes where a center conductor may form at least a part of one electrode and at an outer conductive shield may form at least a part of the other electrode. The catheter 44 and its electrode pair 46 provide shock waves as previously described. Such an arrangement will decalcify the leaflets 18. This not only will decalcify the leaflets 18, but will also soften the aortic valve annulus and expand its diameter. Hence, the balloon 42 provides the added advantage of exerting expansion pressure directly to the annulus of the valve to remodel the annulus diameter.

While particular embodiments of the present invention have been shown and described, modifications may be made, and it is therefore intended to cover all such changes and modifications which fall within the true spirit and scope of the invention.

What is claimed is:

1. An intravascular valvuloplasty system for treating a heart valve having leaflets, the system comprising:
a power supply configured to generate voltage pulses;
an elongate body having an inflation lumen;
a shock wave generator for generating shock waves and comprising an electrode pair connected to the power supply and including a first electrode formed by an outer conductive shield and a second electrode formed by an inner conductor, the electrode pair configured to create shock waves when the voltage pulses are applied to the electrodes; and
a balloon that is inflatable with a liquid via the inflation lumen and is configured to remain intact as the shock waves propagate through the liquid in the balloon.

2. The system of claim 1, wherein the outer conductive shield and the inner conductor are coaxial.

3. The system of claim 1, wherein the outer conductive shield is annular.

4. The system of claim 1, wherein the shock wave generator is configured to create the shock waves to impinge on the leaflets.

5. The system of claim 1, wherein the power supply is configured to generate voltage pulses that are synchronized to R waves of the heart.

6. The system of claim 1, wherein the balloon has a reduced diameter portion configured to be received within a valve annulus.

7. The system of claim 1, wherein the outer conductive shield is insulated from the inner conductor.

8. An intravascular valvuloplasty system for treating a heart valve having leaflets, the system comprising:
a power supply configured to generate voltage pulses;
an elongate body having an inflation lumen;
a first shock wave generator comprising a first electrode pair connected to the power supply and including a first inner electrode and a first outer electrode that is disposed outwardly of the first inner electrode;
a second shock wave generator comprising a second electrode pair connected to the power supply and including a second inner electrode and a second outer electrode that is disposed outwardly of the second inner electrode;
a balloon that is inflatable with a liquid via the inflation lumen and is configured to remain intact as the shock waves propagate through the liquid in the balloon,
where the first and second shock wave generators are each configured to create shock waves when the voltage pulses are applied to the first and second electrodes.

9. The system of claim 8, wherein the first inner electrode and first outer electrode are coaxial and the second inner electrode and the second outer electrode are coaxial.

10. The system of claim 8, wherein the first and second shock wave generators are configured to create shock waves concurrently to impinge on the leaflets.

11. The system of claim 8, wherein the first shock wave generator is configured to be positioned on a first side of the leaflets and the second shock wave generator is configured to be positioned on a second side of the leaflets.

12. The system of claim 8, wherein the first shock wave generator is longitudinally spaced from the second shock wave generator.

13. The system of claim 8, wherein the balloon has a reduced diameter portion configured to be received within a valve annulus.

14. The system of claim 8, wherein the balloon has a reduced diameter portion and the first and second shock wave generators are positioned in opposite sides of the reduced diameter portion of the balloon.

15. The system of claim 8, wherein the first inner electrode is insulated from the first outer electrode and the second inner electrode is insulated from the second outer electrode.

16. The system of claim 8, wherein the balloon comprises a first chamber surrounding the first shock wave generator and a second chamber surrounding the second shock wave generator.

17. The system of claim 16, wherein the inflation lumen is configured to inflate both the first chamber and the second chamber.

18. The system of claim 16, wherein the inflation lumen is configured to inflate only one of the first chamber and the second chamber.

19. The system of claim 16, wherein first chamber and the second chamber are longitudinally offset from each other.

20. The system of claim 8, wherein the first inner electrode is located at a distal end of a first conductor that is connected to the power supply and the second inner electrode is located at a distal end of a second conductor that is connected to the power supply.
